(19) United States
(12) Patent Application Publication
(10) Pub. No.: US 2022/0257365 A1
(43) Pub. Date: Aug. 18, 2022

(54) IMPLANT FOR RECONSTRUCTION OF NIPPLE-AREOLA COMPLEX, AND METHOD FOR PRODUCING SAME

(71) Applicants: T&R BIOFAB CO., LTD., Siheung-si (KR); THE CATHOLIC UNIVERSITY OF KOREA INDUSTRY-ACADEMIC COOPERATION FOUNDATION, Seoul (KR)

(72) Inventors: Da Mi CHOI, Seongnam-si (KR); Jeong Seok LEE, Seongnam-si (KR); Jin Hyung SHIM, Siheung-si (KR); Won Soo YUN, Siheung-si (KR); Jung Ho LEE, Seoul (KR)

(21) Appl. No.: 17/610,132

(22) PCT Filed: Mar. 20, 2020
(86) PCT No.: PCT/KR2020/003863

§ 371 Date: Nov. 9, 2021

(30) Foreign Application Priority Data
May 13, 2019 (KR) 10-2019-0055763

(51) Int. Cl. A61F 2/12; A61F 2/00; A61L 27/14; A61L 27/56; B29C 64/118; B29C 64/209

(52) U.S. Cl. CPC A61F 2/12; A61F 2/0059; A61F 2/0063; B33Y 10/00; A61L 27/56; B29C 64/118; B29C 64/209; A61L 27/14

(57) ABSTRACT

The present invention relates to: an implant for the reconstruction of the nipple-areola complex; and a method for producing same. The implant for the reconstruction of the nipple- areola complex according to the present invention can be implanted along a central axis inside autologous tissue during nipple-areola complex reconstruction surgery to form a normal nipple-areola complex shape. In addition, the nipple-areola complex implant according to the present invention, due to being formed of a porous structure, has similar flexibility to normal nipple-areola complex tissue, and exhibits a high engraftment rate with autologous tissues since the surrounding tissue can permeate into the implant through the voids thereof following implantation.

### DESCRIPTION

Technical Field

The present disclosure relates to an implant for reconstruction of the nipple-areola complex (NAC) and a method for producing the same. More particularly, the present disclosure relates to an implant for reconstruction of the nipple-areola complex (NAC) and a method for producing the same, in which the implant can be implanted along the central axis of the breast inside autologous tissue during nipple-areola complex reconstruction surgery to reconstruct the nipple-areola complex to a near-normal shape.

Background Art

The reconstruction of the nipple-areola complex (NAC) is the final step of breast reconstruction. Although the reconstruction is a simple surgery, it results in high aesthetic and psychological satisfaction of patients.

Patients who need reconstruction of the NAC have congenital factors and acquired factors. Most of the acquired factors are caused by total mastectomy following malignant tumor removal such as breast cancer.

In particular, NAC reconstruction for breast cancer patients is the final step in the long journey of diagnosis and treatment of breast cancer and reconstruction, and thus plays an important aesthetic and psychological role in treating patients. Therefore, bilateral symmetry of the NAC is the primary goal of NAC reconstruction. To achieve this, it is vital to maintain long-term nipple projection.

As a conventional surgical method for NAC reconstruction, implantation of autologous tissue (costal cartilage, fat, etc.) has been used. The method using autologous tissue is a relatively excellent surgical method in terms of the projection, shape, and texture of the nipple, but it can be performed only when the size of a normal nipple on the opposite side is large. In addition, scars are left in a donor site, and the implanted autologous tissue is difficult to engraft into the skin and the nipple when the blood circulation in the nipple is poor, resulting in tissue necrosis.

Another surgical method for NAC reconstruction is to use the C-V flap technique in which, as illustrated in FIG. 1, a plate- shaped acellular dermal matrix is rolled and placed into a breast pocket along the central axis of the breast and used for the maintenance of nipple projection.

In the case of the method using the acellular dermal matrix, absorption of the acellular dermal matrix leads to a reduction in height of an operated nipple, and the use of the acellular dermal matrix is limited in reproducing the appearance of a normal nipple, thereby reducing the aesthetic and psychological satisfaction of patients.

Disclosure

Technical Problem

An objective of the present disclosure is to provide an implant for reconstruction of the nipple-areola complex (NAC) and a method for producing the same, in which the implant can be implanted along the central axis of the breast inside autologous tissue during nipple-areola complex reconstruction surgery, thereby reconstructing the nipple-areola complex to a near normal shape while maintaining the shape thereof in a patient's body for a long period of time.

Another objective of the present disclosure is to provide an implant for reconstruction of the nipple-areola complex (NAC) and a method for producing the same, in which the implant can reconstruct the nipple-areola complex to a near normal shape which gives patients high satisfaction in terms of aesthetics and psychological aspects.

Technical Solution

An aspect of the present disclosure provides an implant for reconstruction of the nipple-areola complex. The implant may include a cylindrical body having a porous structure.

The body may have a structure in which microfibers are stacked at regular intervals. The body may be provided with a hole extending from top to bottom of the body.

Furthermore, the implant may further include a disc- shaped support provided at a lower end of the body. The support may have a porous structure in which microfibers are stacked at regular intervals.

Each of the body and the support may include at least one selected from the group consisting of poly-lactic acid (PLA), poly-glycolic acid (PGA), polycaprolactone (PCL), poly-lactic-co- glycolic acid (PLGA), polyurethane (PU), and polyethylene (PE).

The body may have a diameter of 5 to 25 mm. The microfibers of the body may have a diameter of 100 to 700 μm, and the microfibers of the body may have an interval of 50 to 1500 μm.

Furthermore, the body may have a porosity of 45% to 75%.

The microfibers of the support may have a diameter of 20 to 200 μm. The support may have a porosity of 60% to 75%.

Another aspect of the present disclosure provides a method of producing an implant for reconstruction of the nipple- areola complex. The method may include: injecting a polymer into a syringe of a 3D printer head and heating the syringe to melt the polymer; and extruding the molten polymer through the 3D printer head to form a cylindrical body having a porous structure.

The polymer may be at least one selected from the group consisting of poly-lactic acid (PLA), poly-glycolic acid (PGA), polycaprolactone (PCL), poly-lactic-co-glycolic acid (PLGA), polyurethane (PU), and polyethylene (PE).

Furthermore, the method may further include, after the heating of the syringe, extruding a polymer through the 3D printer head to form a disc-shaped support (200) having a porous structure.

The body may have a porosity of 45% to 75%. The support may have a porosity of 60% to 75%.

Advantageous Effects

Conventionally, it was impossible to reconstruct the nipple-areola complex in a form resembling that of a normal nipple because nipple-areola complex reconstruction surgery was performed using a planar plate-shaped acellular dermal layer or an amorphous filler which are not in a form of a normal nipple-areola complex.

However, since an implant for reconstruction of the nipple-areola complex according to the present disclosure has a form resembling that of the normal nipple-areola complex, it is possible to implant the implant according to the present disclosure along the central axis of the breast inside autologous tissue during the nipple-areola complex reconstruction surgery, thereby restoring the nipple-areola complex to a near normal shape which gives patients high aesthetic and psychological satisfaction.

In addition, the implant for reconstruction of the nipple-areola complex according to the present disclosure can maintain the shape thereof in a patient's body for a long period of time after implantation and thus is effective in reconstructing the nipple-areola complex.

In addition, the implant for reconstruction of the nipple-areola complex according to the present disclosure has a porous structure and thus can exhibit a flexibility resembling that of normal nipple-areola complex tissue. The implant can also exhibit a high engraftment rate with autologous tissues because surrounding tissue can permeate into the implant through the voids thereof following implantation.

### Description of Drawings

FIG. 1 is a schematic view schematically illustrating a C-V flap technique.

FIG. 2 is a perspective view illustrating an implant for reconstruction of the nipple-areola complex according to an embodiment of the present disclosure.

FIG. 3 is a front view illustrating the implant for reconstruction of the nipple-areola complex according to the embodiment of the present disclosure.

FIG. 4 is a perspective view illustrating an implant for reconstruction of the nipple-areola complex according to another embodiment of the present disclosure.

FIG. 5 is a front view illustrating the implant for reconstruction of the nipple-areola complex according to the other embodiment of the present disclosure.

FIG. 6 is a product image of the implant for reconstruction of the nipple-areola complex according to the embodiment of the present disclosure.

FIG. 7 illustrates images of the results of evaluating ease of tissue penetration into the implant for reconstruction of the nipple-areola complex according to the embodiment of the present disclosure over time.

FIG. 8 illustrates images of the results of measuring a cell growth rate as a function of a change in porosity of the implant.

FIG. 9 is a graph illustrating the results of measuring a compressive strength as a function of the porosity of the implant for reconstruction of the nipple-areola complex according to the embodiment of the present disclosure.

FIG. 10 and FIG. 11 illustrate the results of measuring an increase in projection as a result of implantation of the implant for reconstruction of the nipple-areola complex according to the embodiment of the present disclosure.

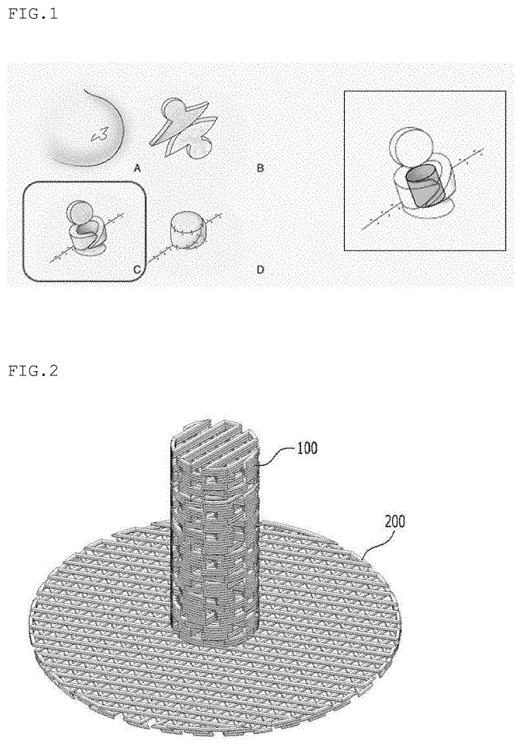

Mode for Invention

Reference will now be made in detail to exemplary embodiments of the present disclosure. All terms or words used herein should not be interpreted as being limited merely to common and dictionary meanings but should be interpreted as having meanings and concepts which are defined within the technical scope of the present disclosure.

In the specification, when a part is referred to as “including” an element, this means that the part does not exclude another element and may further include another element unless stated otherwise.

Hereinafter, an implant for reconstruction of the nipple-areola complex (NAC) according to the present disclosure and a method for producing the same will be described in more detail.

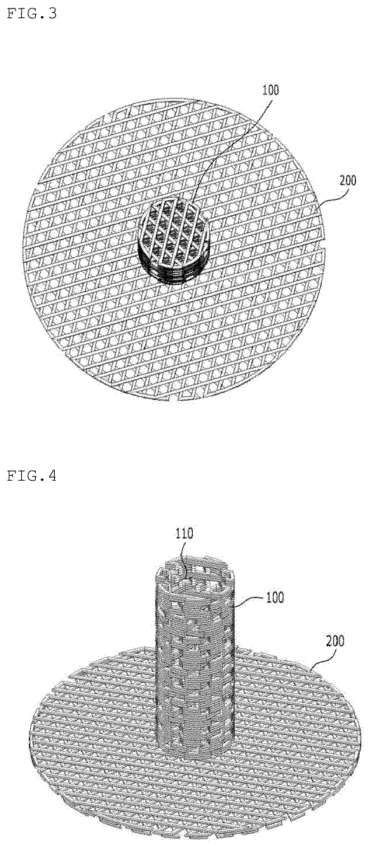

FIG. 2 is a perspective view illustrating an implant for reconstruction of the nipple-areola complex according to an embodiment of the present disclosure, and FIG. 3 is a front view illustrating the implant for reconstruction of the nipple-areola complex according to the embodiment of the present disclosure.

Referring to FIGS. 2 and 3, the implant for reconstruction of the nipple-areola complex according to the embodiment of the present disclosure may include a cylindrical body 100 having a porous structure, and if necessary, may further include a disc-shaped support 200 provided at a lower end of the body 100.

Preferably, each of the body 100 and the support 200 has a porous structure in which microfibers are stacked at regular intervals to form voids. The voids formed as a result of stacking the microfibers is not particularly limited in shape. For example, the voids may have a polygonal shape such as a triangle or a lattice depending on the cross direction of the microfibers.

With such a porous structure, the implant for reconstruction of the nipple-areola complex according to the present disclosure exhibits a flexibility resembling that of normal nipple-areola complex tissue, and exhibits a high engraftment rate with autologous tissues because surrounding tissue can permeate into the implant through the voids thereof following implantation.

The body 100 serves to reconstruct the nipple area of the breast of a patient by maintaining the shape and height of the nipple, and may be formed by stacking microfibers dozens of times.

Preferably, the porosity of the body 100 is about 45% to 75%. When the porosity thereof is less than 45%, it is difficult for the surrounding tissue to penetrate into the implant for reconstruction of the nipple-areola complex after implantation. On the other hand, when the porosity thereof exceeds 75%, the strength of the implant decreases, and thus it cannot maintain the height of the nipple.

The porosity of the body 100 is determined by the diameter of the microfibers of the body 100 and the interval between the microfibers. Preferably, the diameter of the microfibers is about 100 to 700 μm, and the interval between the microfibers is about 50 to 1500 μm. The interval between the microfibers may be regular or irregular. For example, the interval between the microfibers may be configured to be narrowed or widened depending on the positions of the microfibers. In another example, the interval may be configured to be increased or decreased at a predetermined rate (these exemplary methods may be applied in the same manner to the microfibers constituting the support which will be described later).

When the diameter of the microfibers is less than 100 μm, the size of the voids of the body 100 becomes too small, which hinders the penetration of the surrounding tissue into the implant for reconstruction of the nipple-areola complex after implantation.

The height and diameter of the body 100 may vary depending on the shape of a nipple to be reconstructed. Preferably, the diameter of the body 100 is about 5 to 25 mm.

As illustrated in FIGS. 4 and 5, the body 100 may have a hollow porous structure with holes 110 extending from top to bottom of the body 100. Because the body 100 of hollow porous structure has an empty interior space, this is advantageous in that more autologous tissues (new tissues including soft tissue and surrounding adipose tissue) can quickly penetrate into the interior space after implantation.

In addition, an acellular dermal matrix may be inserted into the empty interior space of the hollow body 100, so that the shape of the acellular dermal matrix may be maintained while the strength of the hollow body 100 may be increased.

The support 200 serves to provide convenience during implantation by precisely positioning the body 100 on the nipple area of the breast to be reconstructed and maintaining the central axis of the breast, and to reconstruct the areola area of the breast.

The support 200 may be formed by stacking about 1 to 10 layers of microfibers having a diameter of about 50 to 200 μm. Preferably, the height of the support 200 is about 1 to 2 mm, and the porosity of the support 200 is about 60% to 75%.

Preferably, the porosity of the support 200 is different from that of the body 100 described above, and more preferably, the porosity of the support 200 is larger than that of the body.

The body 100 and the support 200 may be made of a biodegradable polymer. The material thereof may be determined according to application purposes. For example, a non-degradable polymer, or a polymer composite being a mixture of the biodegradable polymer and the non-degradable polymer may be used. For example, each of the body 100 and the support 200 may include at least one selected from the group consisting of poly-lactic acid (PLA), poly- glycolic acid (PGA), polycaprolactone (PCL), poly-lactic-co- glycolic acid (PLGA), polyurethane (PU) and, polyethylene (PE). Of these, preferred is polycaprolactone (PCL).

Polycaprolactone (PCL) is a material with excellent biocompatibility and biodegradability and is harmlessly degraded in the human body over 2 to 3 years by hydrolysis into water and carbon dioxide. When the body 100 and the support 200 are made of such polycaprolactone (PCL), the implant for reconstruction of the nipple-areola complex can maintain its initial shape for more than 12 months after implantation into a body of the patient, thus being effective in reconstructing the nipple-areola complex.

The above-mentioned polymer components may be mixed in an appropriate ratio to properly control the degradation rate of the implant for reconstruction of the nipple-areola complex.

In addition, each of the body 100 and the support 200 may further include hydroxyapatite, tri-calcium phosphate, collagen, and the like which facilitate rapid penetration of the surrounding tissue into the implant for reconstruction of the nipple-areola complex after implantation.

On the other hand, another embodiment of the present disclosure relates to a method for producing an implant for reconstruction of the nipple-areola complex. The method includes: injecting a polymer into a syringe of a 3D printer head and heating the syringe to melt the polymer; and extruding the molten polymer through the 3D printer head to form a cylindrical body 100 having a porous structure. With the method for producing the implant for reconstruction of the nipple-areola complex according to the present disclosure, it is possible to produce an implant for reconstruction of the nipple-areola complex with a high engraftment rate with autologous tissues.

Specifically, the polymer is injected into the syringe of the 3D printer head. In this case, the polymer is preferably at least one selected from the group consisting of poly-lactic acid

(PLA), poly-glycolic acid (PGA), polycaprolactone (PCL), poly- lactic-co-glycolic acid (PLGA), polyurethane (PU), and polyethylene (PE).

In addition, hydroxyapatite, tri-calcium phosphate, collagen, and the like may be added to the polymer to facilitate rapid penetration of surrounding tissue into the implant for reconstruction of the nipple-areola complex after implantation.

After the polymer is injected into the syringe of the 3D printer head, the syringe of the 3D printer head is heated to a temperature at which the polymer is melted for 3D printing. For example, when the polymer is polycaprolactone (PCL), the syringe of the 3D printer head is heated to a temperature of 90 to 150° C.

The molten polymer is extruded through the 3D printer head and stacked at regular intervals in the form of microfibers, resulting in the formation of the porous cylindrical body 100.

At this point, the polymer may be discharged by means of pneumatic pressure or the like. When the polymer is polycaprolactone (PCL), the polymer is preferably discharged under a pressure of 60 to 110 kPa.

In addition, the polymer is preferably discharged so that the diameter of the microfibers of the body 100 is about 100 to 700 μm and the interval between the microfibers of the body 100 is about 50 to 1500 μm.

When the diameter of the microfibers is less than 100 μm, the size of the voids of the body becomes too small, which hinders the penetration of the surrounding tissue into the implant for reconstruction of the nipple-areola complex after implantation.

In addition, the porous cylindrical body 100 is preferably formed to have a porosity of about 45% to 75%. When the porosity thereof is less than 45%, it is difficult for the surrounding tissue to penetrate into the implant for reconstruction of the nipple-areola complex after implantation. On the other hand, when the porosity thereof exceeds 75%, the strength of the implant decreases, and thus it cannot maintain the height of the nipple.

In addition, the method for producing the implant for reconstruction of the nipple-areola complex according to the present disclosure may further include, after the heating of the syringe, extruding a polymer through the 3D printer head to form a disc-shaped support 200 having a porous structure.

The forming of the disc-shaped support 200 may be performed in the following manner. First, the polymer is injected into the syringe of the 3D printer head. The syringe of the 3D printer head is then heated to a temperature at which the polymer is melted for 3D printing. Finally, the molten polymer is extruded through the 3D printer head and stacked at regular intervals in the form of microfibers, thereby forming the porous disc-shaped support 200.

In addition, the polymer is preferably discharged so that the diameter of the microfibers of the support 200 is about 50 to 200 μm. The support 200 is preferably formed to have a porosity of about 60% to 75%.

Hereinafter, examples of the present disclosure will be described. However, the scope of the present disclosure is not limited to the following examples. Accordingly, those skilled in the art will appreciate that various modifications, additions, and substitutions are possible, without departing from the scope and spirit of the present disclosure as disclosed in the accompanying claims.

[Example 1]

An implantation sample (10 mm x 40 mm x 0.8 mm) was prepared using 3D printing. Specifically, an implantation sample having a porosity of 50% was prepared by stacking layers of microfibers having a diameter of 500 μm at intervals of 500 μm by extruding polycaprolactone (PCL) through a 3D printer head.

[Example 2]

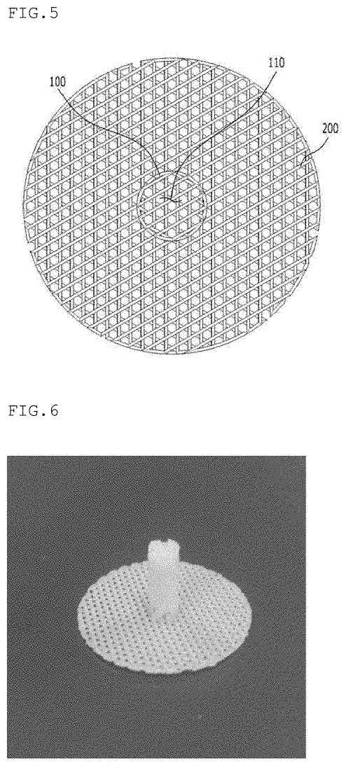

An implant for reconstruction of the nipple-areola complex was produced using 3D printing. Specifically, a support was formed by stacking layers of microfibers having a diameter of 500 μm in a disc shape by extruding polycaprolactone (PCL) through a 3D printer head. The same procedure was then performed to form a cylindrical body on an upper end of the support. As a result, the implant for reconstruction of the nipple-areola complex was produced. To obtain a 50% porosity of the implant for reconstruction of the nipple-areola complex, the interval between the microfibers was set to 500 μm. The 3D printing was performed under conditions in which the diameter of the body was set to 10 mm and the height thereof was set to 5 mm. The implant for reconstruction of the nipple-areola complex thus produced is illustrated in FIG. 6.

[Experimental Example 1:Evaluation of ease of tissue penetration]

Implantation samples prepared according to Example 1 were implanted into the subcutis of rabbits, and the degree of penetration of biological tissue into the implantation samples for 12 months was evaluated. The results are illustrated in FIG. 7.

The results of FIG. 7 revealed that the biological tissue penetrated between the voids of the implantation samples, and the volume of the implantation samples was maintained even when 12 months passed after implantation.

[Experimental Example 2: Measurement of cell growth rate as function of porosity]

In order to confirm a tissue regeneration effect as a function of porosity, human fibroblasts were cultured for 14 days in samples having different porosities. The samples having different porosities were prepared in the same manner as in Example 1 so as to have porosities of 30%, 50%, 70%, and 80%, respectively.

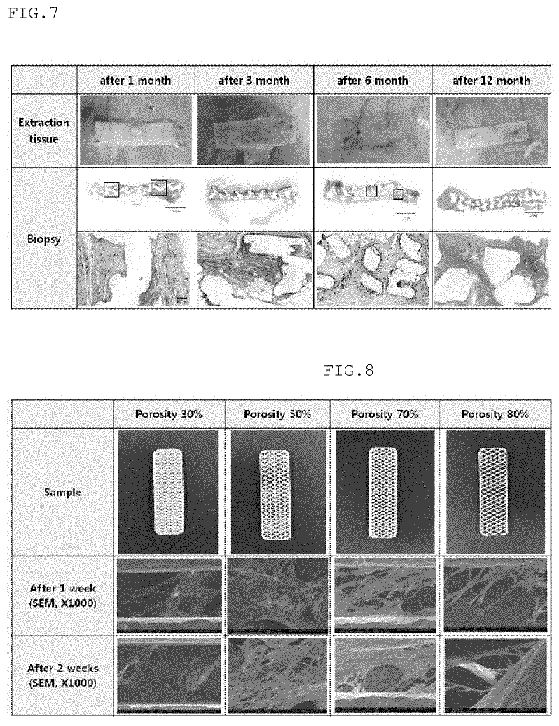

Human fibroblasts with a cell concentration of 1×10⁵ cells/ml were seeded in each sample and then cultured in Dulbecco's Modified Eagle Medium (DMEM, Gibco®) (37° C., 5% CO₂). The engraftment and growth rates of the human fibroblasts of each sample were observed using a field-emission scanning electron microscope (FE-SEM, S-4700, HITACHI Co, Japan). The results are illustrated in FIG. 8.

The results of FIG. 8 revealed that in the case of the sample having a porosity of 30%, this low porosity made it difficult to achieve rapid cell penetration, and resulted in an insufficient contact area for cell growth, so that cell growth was hindered. In addition, in the case of a sample having a porosity of 80%, this high porosity resulted in an insufficient engraftment surface on which cells could engraft and grow, so that cell growth was slowed down. On the other hand, in the case of samples having porosities of 50% and 70%, rapid cell penetration was achieved while a sufficient engraftment surface for cell growth was provided, so that a high cell growth rate was yielded.

[Experimental Example 3: Measurement of compressive strength as function of porosity]

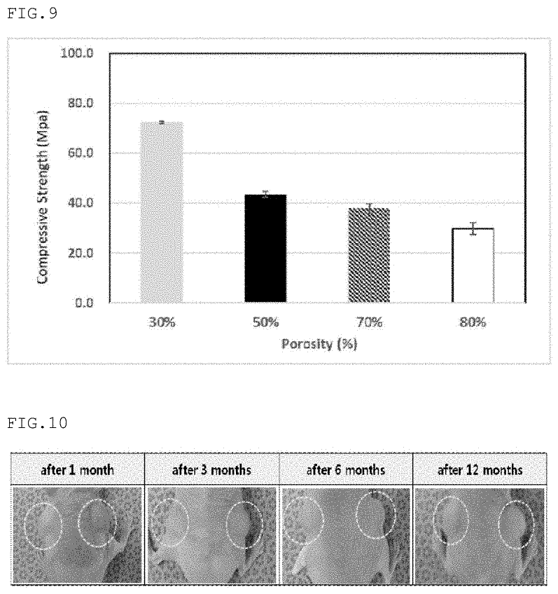

The implant for reconstruction of the nipple-areola complex needs to able to withstand a pressure caused by external resistance and fatigue after implantation, while having a strength resembling that of a normal nipple. To measure a compressive strength as a function of the porosity of the implant, strength measurement samples were prepared in the same manner as the body of Example 2 so as to have different porosities (30%, 50%, 70%, and 80%). The compressive strength of each of the strength measurement samples having different porosities was measured using a universal testing machine (Instron, Cat No. 3343) in accordance with the standard of ISO 604-PLASTICS-DETERMINATION OF COMPRESSIVE PROPERTIES. The results are illustrated in FIG. 9.

The results of FIG. 9 revealed that when the porosity was 30%, the compressive strength was 72.5 Mpa, which was higher than that of the normal nipple. In addition, when the porosity was 80%, the compressive strength was 30 Mpa, which was very low and not sufficient to withstand external resistance. On the other hand, when the porosity was 50% and 70%, the compressive strength was 48 to 50 Mpa, which was similar to that of the normal nipple.

[Experimental Example 4: Implant implantation]

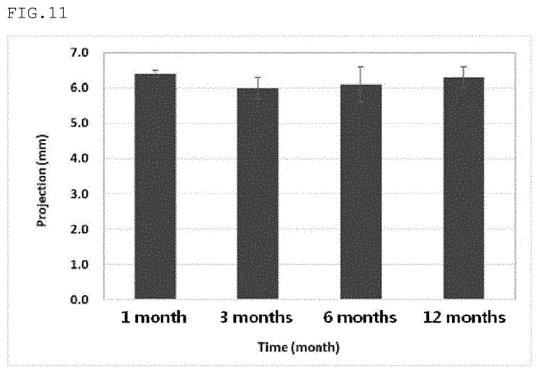

After the full-thickness skin of nude mice (Male, BALB/c nude mice, 20 to 25 mg) was incised to a length of about 10 mm, implants for reconstruction of the nipple-areola complex prepared according to Example 2 were implanted into the subcutis of the mice. After implantation, images of the mice with a projected appearance as a result of implantation of the implant for reconstruction of the nipple-areola complex were captured and are illustrated in FIG. 10. An increase in projection (from the skin layer at a normal site) as a result of implantation of the implant for reconstruction of the nipple-areola complex was measured with digital vernier calipers (Mitutoyo, Cat No. 500-151-30). The results are illustrated in FIG. 11 (measurement was made at 1 month, 3 months, 6 months, and 12 months after implantation).

The results of FIGS. 10 and 11 revealed that the increase in projection as a result of implantation of the implant for reconstruction of the nipple-areola complex was maintained at equal to or greater than 6 mm for 12 months after implantation. This indicated that the shape of the implant for reconstruction of the nipple-areola complex was maintained as surrounding tissue penetrated into the implant through the voids thereof and new tissue regeneration was achieved.

Industrial Applicability

The present disclosure provides an implant for reconstruction of the nipple-areola complex and a method for producing the same, in which the implant can be implanted along the central axis of the breast inside autologous tissue during nipple- areola complex reconstruction surgery, thereby reconstructing the nipple-areola complex to a near normal shape while maintaining the shape thereof in a patient's body for a long period of time. Since the implant for reconstruction of the nipple-areola complex according to the present disclosure can be implanted along the central axis of the breast inside autologous tissue during nipple- areola complex reconstruction surgery, it is possible to reconstruct the nipple-areola complex to a near normal shape which gives patients high aesthetic and psychological satisfaction. After implantation, the implant can maintain the shape thereof in the patient's body for a long period of time and thus is effective in reconstructing the nipple-areola complex. Therefore, the present disclosure has industrial applicability.

1. An implant for reconstruction of the nipple-areola complex, the implant comprising a cylindrical body (100) having a porous structure,
wherein the body (100) has a structure in which microfibers are stacked at regular intervals.

2. The implant of claim 1, wherein the body (100) is provided with a hole (110) extending from top to bottom of the body (100).

3. The implant of claim 1, further comprising a disc-shaped support (200) provided at a lower end of the body (100), wherein the support (200) has a porous structure in which microfibers are stacked at regular intervals.

4. The implant of claim 1, wherein the body (100) has a diameter of 5 to 25 mm.

5. The implant of claim 1, wherein the microfibers of the body (100) have a diameter of 100 to 700 μm and the microfibers of the body (100) have an interval of 50 to 1500 μm.

6. The implant of claim 1, wherein the body (100) has a porosity of 45 to 75%.

7. The implant of claim 3, wherein the microfibers of the support (200) have a diameter of 20 to 200 μm.

8. The implant of claim 1, wherein the support (200) has a porosity of 60 to 75%.

9. The implant of claim 1, wherein the body (100) comprises at least one selected from the group consisting of poly-lactic acid, poly-glycolic acid, polycaprolactone, poly-lactic- co-glycolic acid, polyurethane, and polyethylene.

10. The implant of claim 3, wherein the body (100) and the support (200) have different porosities.

11. A method of producing an implant for reconstruction of the nipple-areola complex, the method comprising:
injecting a polymer into a syringe of a 3D printer head and heating the syringe to melt the polymer; and
extruding the molten polymer through the 3D printer head to form a cylindrical body (100) having a porous structure.

12. The implant of claim 11, wherein the polymer is at least one selected from the group consisting of poly-lactic acid, poly-glycolic acid, polycaprolactone, poly-lactic-co-glycolic acid, polyurethane, and polyethylene.

13. The implant of claim 11, wherein the body (100) has a porosity of 45 to 75%.

14. The method of claim 11, further comprising, after the heating of the syringe, extruding a polymer through the 3D printer head to form a disc-shaped support (200) having a porous structure.

15. The implant of claim 14, wherein the support (200) has a porosity of 60 to 75%.

16. The implant of claim 14, wherein the body (100) and the support (200) have different porosities.

17. The implant of claim 2, further comprising a disc-shaped support (200) provided at a lower end of the body (100), wherein the support (200) has a porous structure in which microfibers are stacked at regular intervals.

18. The implant of claim 17, wherein the microfibers of the support (200) have a diameter of 20 to 200 μm.

19. The implant of claim 17, wherein the body (100) and the support (200) have different porosities.
